(19) United States
(12) Patent Application Publication
(10) Pub. No.: US 2008/0248573 A1
(43) Pub. Date: Oct. 9, 2008

(54) Mixture for Oocytes Maturation in Art Laboratory

(76) Inventor: Mohamed Hamdi Hasan Badrawi, Cairo (EG)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/088,185

(22) PCT Filed: Jun. 6, 2006
(86) PCT No.: PCT/EG2006/000020

§ 371 Date: Mar. 26, 2008

(51) Int. Cl. C12N 5/00

(52) U.S. Cl. 435/408

(57) ABSTRACT

Women over 40 years and those who had ovarian problems suffer from low pregnancy rate after Assisted Reproduction Technology (ART) procedure due to immature oocytes. According to the invention a mixture is added to these oocytes. This mixture consists of two substances: Substance 1 which has the ability of stimulating cell proliferation in vitro by increasing the oxygen uptake of the cell and substance 2 which is any of the most used media in ART laboratories. Substance 1 is protein-free haemodialysate of calves blood. The created mixture in more than one concentration succeeded in complete oocytes maturation from immature to complete matured ones, able to be injected and fertilized by sperm. A second aspect of the invention is the use of the mixture to increase motility of sperm by 30% when sperm is incubated before Intrauterine Insemination (IUI).

### TECHNICAL FIELD

In vitro maturation of human oocytes in the laboratory of Assisted Reproduction Technology (ART) prior of performing procedures (IVF/ICSI/IUI . . . etc) leading to fertilization and consequantly occurence of pregnancy.

### BACKGROUND

Women over forty years and some other ovarian diseases suffer from low incedence of pregnancy rate after ICSI (Intra cytoplasmic Sperm Injection) due to the prevalence of immature oocytes collected which fail to be fertilized if injected with sperm in the laboratory.

There is many known media ready made by many manufactures where the oocytes collected are incubated examined and injected with sperm in the laboratory but it has little effect on oocytes maturation.

Many compounds and hormones where added to these media as a trial to create a new mixture which could enhance oocytes maturation before sperm injection but without excellent results.

There is always a need to find a substance which if added to the media used in the lab. for ICSI. Creating a new mixture, which can stimulate the process of oocytes maturation with good results and consequently increase the fertilization and pregnancy rate, will open a new hope for women over 40 years and those who had problem in producing mature oocyte.

### DISCLOSURE OF THE INVENTION

1—At a certain stage during the process of ICSI (Intra Cytoplasmic Sperm Injection) oocyte collection is done after ovarian stimulation.

2—The oocytes were incubated in culture media available in the market and examined by special microscope.

3—Three type of oocytes could be detected:
a—Immature oocytes called GV and it is usually discarded as it does not divide if injected with sperm.
b—Incomplete mature oocytes called metaphase 1 and this if it is left for a certain period in the culture media it may sometime proceed to complete maturation then could be injected with sperm.
c—Complete mature oocytes and these are idial for sperm injection called mataphase 11.

4—In normal young female the third type (comple mature mataphase 11) oocytes are expected to be collected and injected with sperm and all other types of oocytes are excluded.

The problem arise in female after the age of 40 or with special diseases of in the ovaries where collection of immature oocytes are the common findings, which became a problem which causes failure of the procedure to the extent that many of the ART (Assisted Reproduction Technology) centers refuse to accept these women due to the high failure rate.

5—A new culture mixture BE (Badrawi-Egypt) is invented which allows the immature oocytes and the incomplete mature oocytes to complete its maturation within about 24 hours and became able to be injected successfully by sperm.

Adding substance 1 (available in the market) to substant 2 as follows:

a—By adding one micron(=0.0425 mg/ml) of substance 1 to 99 micron of substance 2 (1:99) a new mixture BE1 is created able transfer metaphase 1 (incomple mature) oocytes to metaphase 2 (complete mature) within a period of about 24 hours.

b—By adding five micron of substance 1 (=0.2125 mg/ml) to 95 micron of substance 2 (5:95) a new mixture BE2 is created able to transfer Gv (immature) oocytes to metaphase 2 (complete mature) oocytes over a period about 24 hours.

Also the mixture BE 2 has the ability to increase the sperm motility by 30% if it is used as incubation media prior to Intra Uterine Insemination.

Formula:

a—Substance 1: Protein-free haemodialysate of calves' blood (Bos taurus) chemically and biologically standerdised, 42.5 mg/ml.(market name solcosery 1 made by ICN Swizerland AG, birsfelden, Swizerland)

b—Substance 2: Any media used in ART lab. for incubation of oocytes the one which was tested is composed of Human serum albumin, Glucose and derived metabolites, Physiological salts, Essential amino acids, Non essential amino acids, Vitamins, Nucleotides, Sodium bicarbonates, Streptomycin 40 mg/litre, Penicellin 40000 iu/litre, Phenol red, market name ISM1(Medicult, Denmark).

Utility Use:

THE NEW MIXTURE BE 1 AND 2 COULD INDUSTRIALY MANUFACTURED TO BE USED BY ALL ART CENTER ALL OVER THE WORLD INSTEAD OF THE ORDINARY MEDIA KNOWN TO ALLOW A MARKED IMPROVEMENT IN ICSI PROCEEDURES IN WOMEN ABOVE 38 YEARS AND WOMEN HAVING SOME OVARIAN DISEASES, OR AS A GOOD ALTERNATIVE TO ANY MEDIA TO PROMOTE CELL DIVISION IN ALL ART PROCEDURES.

1- The new mixtures BE1/BE2 (Badrawi-Egypt) with two different concentration made by adding two different substances ready made in the market has the ability to complete oocytes maturation from Gv (immature) to metaphase 1 up to metaphase 2 (mature) within a period around 24 hours.

2- The mixture in claim 1 is formed by adding substance 1 which is: Protein-free haemodialysate of calves' blood (Bos taurus) chemically and biologically standardised, 42.5 mg/ml. (market name solcosery 1 made by ICN Swizerland AG, birsfelden, Swizerland) throught its property of stimulation cell proliferation in vitro by increasing the oxygen uptake of the mitochondria/to substant 2: ISMI Made by Medicult, Denmark.
substance 2 alone is a known culture media used in art laboratory for incubation of oocytes and sperm injection and do not stimulate oocytes maturation.

3- A mixture according to claim 2 having the ability to complete oocytes maturation from immature or incomplete mature to complete mature oocytes within a short period, make them ready for fertilization beside its basic function as incubation media.

4- The mixture BE1 and BE2 stimulate sperm motility when used as mixture to incubate sperms prior to IUI (Intra Uterine Insimination) procedure especialy that the main indication for IUI is low sperm motility.

5- The new mixture opens the field for in vitro research concerning promoting fertilized oocytes devision and its clinical implications. As adding substance 1 (Protein-free haemodialysate of calves' blood) to any media used in art laboratory with the same specification of substance ISMI under any other trade name by same concentration lead to the same results stated above.
